(19) United States
(12) Patent Application Publication
(10) Pub. No.: US 2024/0335440 A1
(43) Pub. Date: Oct. 10, 2024

(54) COMBINATION THERAPY COMPRISING A PKC INHIBITOR AND A C-MET INHIBITOR

(71) Applicants: IDEAYA BIOSCIENCES, INC., South San Francisco, CA (US); PFIZER INC., New York, NY (US)

(72) Inventors: Matthew Anthony MAURER, South San Francisco, CA (US); Michael Gabriel O'QUIGLEY, South San Francisco, CA (US); Richard ZANG, South San Francisco, CA (US); Jaymes HOLLAND, South San Francisco, CA (US); Mai Hope LE, South San Francisco, CA (US); Sarah Shwu-Kuan JAW-TSAI, South San Francisco, CA (US)

(21) Appl. No.: 18/715,927

(22) PCT Filed: Dec. 5, 2022
(86) PCT No.: PCT/US2022/080928

§ 371 Date: Jun. 3, 2024

Related U.S. Application Data
(60) Provisional application No. 63/286,345, filed on Dec. 6, 2021.
(60) Provisional application No. 63/317,573, filed on Mar. 8, 2022.
(60) Provisional application No. 63/370,056, filed on Aug. 1, 2022.

(51) Int. Cl. A61K 31/497; A61K 31/4545; A61P 35/00

(52) U.S. Cl. CPC A61K 31/497; A61K 31/4545; A61P 35/00

(57) ABSTRACT

Provided herein is a combination therapy and methods of using such combination therapy to treat diseases or disorders associated with PKC and c-MET.

### RELATED APPLICATIONS

This application claims priority to U.S. Provisional Application No. 63/286,345, filed Dec. 6, 2021; U.S. Provisional Application No. 63/317,573, filed Mar. 8, 2022; and U.S. Provisional Application No. 63/370,056, filed Aug. 1, 2022, the entire contents of which are incorporated by reference in its entirety.

### BACKGROUND

Uveal melanoma is the most common primary intraocular malignant tumor in adults. Certain protein kinase inhibitors are described in International Publ. Nos. WO 02/38561 and WO 2008/106692. One protein kinase C (PKC) inhibitor, sotrastaurin, has been shown to have activity against certain PKC isototypes and has only recently been shown to selectively inhibit the growth of uveal melanoma cells harboring GNAQ mutations by targeting PKC/ERK1/2 and PKC/NF-xB pathways (see X. Wu, et al., Mol. Cancer Ther., Vol. 11, pages 1905-1914, 2012). However, there still remains an unmet need to provide next generation PKC inhibitors for treating uveal melanoma that have improved efficacy at lower dosage amounts to achieve tumor regression, improved potency, hERG activity, absorption, gastrointestinal tolerance and kinase selectivity. PCT application no. PCT/IB2015/055951 (WO 2016/020864) discloses a number of potent and selective PKC inhibitors.

The c-MET receptor has been shown to be expressed in a number of human cancers. c-MET and its ligand, HGF, have also been shown to be co-expressed at elevated levels in a variety of human cancers (particularly sarcomas). However, because the receptor and ligand are usually expressed by different cell types, c-MET signaling is most commonly regulated by tumor-stroma (tumor-host) interactions. Furthermore, c-MET gene amplification, mutation, and rearrangement have been observed in a subset of human cancers. Families with germline mutations that activate c-MET kinase are prone to multiple kidney tumors as well as tumors in other tissues. Numerous studies have correlated the expression of c-MET and/or HGF/SF with the state of disease progression of different types of cancer (including lung, colon, breast, prostate, liver, pancreas, brain, kidney, ovaries, stomach, skin, and bone cancers). Furthermore, the overexpression of c-MET or HGF have been shown to correlate with poor prognosis and disease outcome in a number of major human cancers including lung, liver, gastric, and breast. c-MET has also been directly implicated in cancers without a successful treatment regimen such as pancreatic cancer, glioma, and hepatocellular carcinoma. PCT application no. PCT/IB2005/002837 (WO 2006/021884) discloses a number of potent and selective c-MET inhibitors.

### SUMMARY

Provided herein is a combination therapy comprising a PKC inhibitor and a c-MET inhibitor. The combination therapy is useful for the treatment of a variety of cancers, including uveal melanoma. The combination therapy is also useful for the treatment of a variety of cancers, including solid tumors harboring GNAQ and/or GNA11 mutations. The combination therapy is also useful for the treatment of any number of PKC-associated and/or c-MET associated diseases.

Thus, in one aspect, provided herein is a method of treating uveal melanoma or a solid tumor harboring GNAQ or GNA11 mutations in a subject in need thereof, comprising administering to the subject a pharmaceutically effective amount of Compound 1:

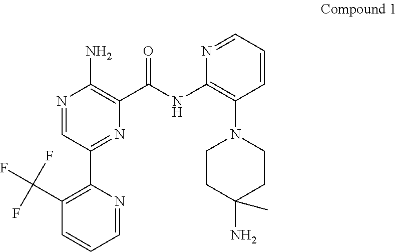 [SMILES: CC1(N)CCN(c2cccnc2NC(=O)c2nc(-c3ncccc3C(F)(F)F)cnc2N)CC1]

or a pharmaceutically acceptable salt thereof,
   and a pharmaceutically effective amount of Compound 2:

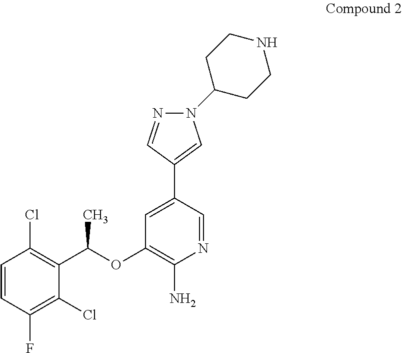 [SMILES: C[C@@H](Oc1cc(-c2cnn(C3CCNCC3)c2)cnc1N)c1c(Cl)ccc(F)c1Cl]

or a pharmaceutically acceptable salt thereof.

In some embodiments, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; wherein
Compound 1, or a pharmaceutically acceptable salt thereof, is administered at a dose of about 200 mg or about 300 mg two times per day (BID) of free base equivalent of Compound 1; and
Compound 2, or a pharmaceutically acceptable salt thereof, is administered at a dose of about 200 mg or about 250 mg BID of free base equivalent of Compound 2.

In some embodiments, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;

wherein
Compound 1, or a pharmaceutically acceptable salt thereof, is administered at a dose of about 300 mg BID of free base equivalent of Compound 1; and
Compound 2, or a pharmaceutically acceptable salt thereof, is administered at a dose of about 200 mg BID of free base equivalent of Compound 2.

In another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;

wherein
Compound 1, or a pharmaceutically acceptable salt thereof, is administered at a dose of about 400 mg to about 600 mg per day (i.e., total daily dose) of free base equivalent of Compound 1; and
Compound 2, or a pharmaceutically acceptable salt thereof, is administered at a dose of about 400 mg to about 500 mg per day of free base equivalent of Compound 2.

In another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;

wherein
Compound 1, or a pharmaceutically acceptable salt thereof, is administered at a dose of about 600 mg per day of free base equivalent of Compound 1; and
Compound 2, or a pharmaceutically acceptable salt thereof, is administered at a dose of about 400 mg per day of free base equivalent of Compound 2.

In some embodiments, the uveal melanoma is metastatic uveal melanoma. The uveal melanoma can be a solid tumor harboring GNAQ or GNA11 mutations.

In some embodiments, the solid tumor harboring GNAQ or GNA11 mutations to be treated is cutaneous melanoma. In another embodiment, the solid tumor harboring GNAQ or GNA11 mutations to be treated is non-small cell lung cancer (NSCLC).

In some embodiments, Compound 1, or a pharmaceutically acceptable salt thereof, is administered at a dose of 300 mg BID. In some embodiments, Compound 1, or a pharmaceutically acceptable salt thereof, is administered at a dose of 300 mg BID of free base equivalent of Compound 1. In another embodiment, Compound 2, or a pharmaceutically acceptable salt thereof, is administered at a dose of 200 mg BID of free base equivalent of Compound 2.

In some embodiments, Compound 1, or a pharmaceutically acceptable salt thereof, is administered at a dose of 200 mg BID of free base equivalent of Compound 1, and Compound 2, or a pharmaceutically acceptable salt thereof, is administered at a dose of 200 mg BID of free base equivalent of Compound 2.

In some embodiments, Compound 1, or a pharmaceutically acceptable salt thereof, is administered at a dose of 300 mg BID of free base equivalent of Compound 1, and Compound 2, or a pharmaceutically acceptable salt thereof, is administered at a dose of 250 mg BID of free base equivalent of Compound 2.

In some embodiments, provided herein is a method of treating uveal melanoma or a solid tumor harboring GNAQ or GNA11 mutations in a subject according to a dosing schedule comprising
(i) a first treatment cycle of at least one 7-day dosing cycle, wherein Compound 1, or an equivalent dose of a pharmaceutically acceptable salt thereof, is administered alone at a dose of about 300 mg BID every day of the first treatment cycle followed by
(ii) a second treatment cycle of at least one 7-day dosing cycle, wherein Compound 1, or an equivalent dose of a pharmaceutically acceptable salt thereof, is administered at a dose of about 300 mg BID every day of the second treatment cycle and Compound 2, or an equivalent dose of a pharmaceutically acceptable salt thereof, is administered at a dose of about 200 mg BID every day of the second treatment cycle.

In some embodiments, the method of treating uveal melanoma or a solid tumor harboring GNAQ or GNA11 mutations in a subject is according to a dosing schedule comprising
(i) a first treatment cycle of at least one 7-day dosing cycle, wherein Compound 1, or an equivalent dose of a pharmaceutically acceptable salt thereof, is administered alone at a dose of about 300 mg BID every day of the first treatment cycle followed by
(ii) a second treatment cycle of at least three 7-day dosing cycles, wherein Compound 1, or an equivalent dose of a pharmaceutically acceptable salt thereof, is administered at a dose of about 300 mg BID every day of the second treatment cycle and Compound 2, or an equivalent dose of a pharmaceutically acceptable salt thereof, is administered at a dose of about 200 mg BID every day of the second treatment cycle.

In yet another embodiment, the method of treating uveal melanoma or a solid tumor harboring GNAQ or GNA11 mutations in a subject is according to a dosing schedule comprising at least one 7-day dosing cycle, wherein Compound 1, or an equivalent dose of a pharmaceutically acceptable salt thereof, is administered at a dose of about 300 mg BID every day of the at least one 7-day dosing cycle and Compound 2, or an equivalent dose of a pharmaceutically acceptable salt thereof, is administered at a dose of about 200 mg BID every day of the at least one 7-day dosing cycle.

In yet another embodiment, the method of treating uveal melanoma or a solid tumor harboring GNAQ or GNA11 mutations in a subject is according to a dosing schedule comprising at least four 7-day dosing cycles, wherein Compound 1, or an equivalent dose of a pharmaceutically acceptable salt thereof, is administered at a dose of about 300 mg BID every day of the at least four 7-day dosing cycles and Compound 2, or an equivalent dose of a pharmaceutically acceptable salt thereof, is administered at a dose of about 200 mg BID every day of the at least four 7-day dosing cycles.

In yet another embodiment, the method of treating uveal melanoma or a solid tumor harboring GNAQ or GNA11 mutations in a subject is according to a dosing schedule comprising at least one 7-day dosing cycle, Compound 1, or an equivalent dose of a pharmaceutically acceptable salt thereof, is administered alone at a dose of about 300 mg BID.

In still another embodiment, the administration of Compound 1, or an equivalent dose of a pharmaceutically acceptable salt thereof, in combination with the administration of Compound 2, or an equivalent dose of a pharmaceutically acceptable salt thereof, is continued uninterrupted during a dosing schedule comprising at least one 7-day dosing cycle.

In some embodiments, Compound 1 and Compound 2 are administered continuously (i.e., a continuous treatment until termination).

With respect to any of the aforementioned embodiments, the method of treating uveal melanoma or a solid tumor harboring GNAQ or GNA11 mutations in a subject in need thereof can further comprise: continuing to administer Compound 1, or an equivalent dose of a pharmaceutically acceptable salt thereof, and Compound 2, or an equivalent dose of a pharmaceutically acceptable salt thereof, uninterrupted during a dosing schedule comprising at least two or more consecutive 7-day dosing cycles, or preferably at least four or more consecutive 7-day dosing cycles. In particularly preferred embodiments, Compound 1, or an equivalent dose of a pharmaceutically acceptable salt thereof, and Compound 2, or an equivalent dose of a pharmaceutically acceptable salt thereof, are continued to be co-administered uninterrupted during a dosing schedule comprising at least eight or more, twelve or more, twenty-four or more, forty-eight or more or ninety-six or more consecutive 7-day dosing cycles.

With respect to any of the aforementioned embodiments, the method of treating uveal melanoma or a solid tumor harboring GNAQ or GNA11 mutations in a subject in need thereof can further comprise: continuing to administer Compound 1, or an equivalent dose of a pharmaceutically acceptable salt thereof, and Compound 2, or an equivalent dose of a pharmaceutically acceptable salt thereof, during a dosing schedule comprising at least two or more consecutive 7-day dosing cycles, or preferably at least four or more consecutive 7-day dosing cycles. In particularly preferred embodiments, Compound 1, or an equivalent dose of a pharmaceutically acceptable salt thereof, and Compound 2, or an equivalent dose of a pharmaceutically acceptable salt thereof, are continued to be co-administered uninterrupted during a dosing schedule comprising at least eight or more, twelve or more, twenty-four or more, forty-eight or more or ninety-six or more consecutive 7-day dosing cycles. In particularly preferred embodiments, Compound 1, or an equivalent dose of a pharmaceutically acceptable salt thereof, and Compound 2, or an equivalent dose of a pharmaceutically acceptable salt thereof, are continued to be co-administered uninterrupted during a dosing schedule comprising at least four or more, eight or more, twelve or more, sixteen or more, twenty or more, twenty-four or more, twenty eight or more, thirty two or more, thirty six or more, forty or more, forty four or more, forty-eight or more, or ninety-six or more, consecutive 7-day dosing cycles.

In another aspect, provided herein is a combination product comprising
(i) about 300 mg Compound 1, or an equivalent dose of a pharmaceutically acceptable salt thereof; and
(ii) about 200 mg of Compound 2, or an equivalent dose of a pharmaceutically acceptable salt thereof.

In yet another embodiment, provided herein is a combination product comprising
(i) about 200 mg to about 300 mg Compound 1, or an equivalent dose of a pharmaceutically acceptable salt thereof; and
(ii) about 200 mg to about 250 mg of Compound 2, or an equivalent dose of a pharmaceutically acceptable salt thereof.

In an embodiment, the combination product comprises Compound 1 as a free base. In another embodiment, the combination product comprises Compound 2 as a free base.

### BRIEF DESCRIPTION OF THE DRAWINGS

FIG. 1: CT scan of a patient with metastatic uveal melanoma (MUM) having diffuse disease in liver, lung, lymph nodes, subcutaneous lesions, and elevated LDH. The patient was dosed with Compound 1 at 300 mg BID (of free base equivalent of Compound 1) and Compound 2 at 200 mg BID (of free base equivalent of Compound 2). The patient was on treatment for approximately 23 weeks.

FIG. 2: CT scan of a patient with MUM having numerous liver lesions and normal LDH. The patient was dosed with Compound 1 at 300 mg BID (of free base equivalent of Compound 1) and Compound 2 at 200 mg BID (of free base equivalent of Compound 2). The patient was on treatment for approximately 12 weeks.

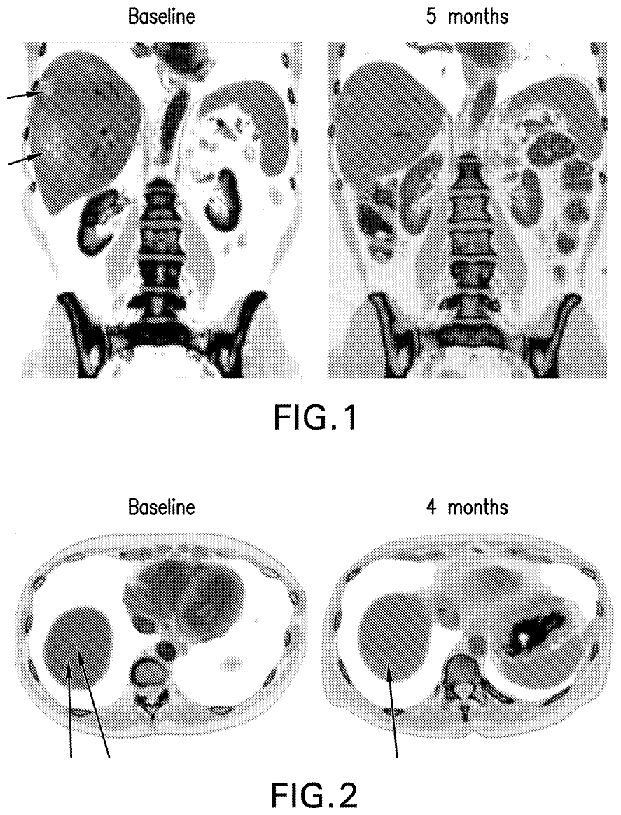

### DETAILED DESCRIPTION

Provided herein is a combination therapy comprising Compound 1, or a pharmaceutically acceptable salt thereof, and Compound 2, or a pharmaceutically acceptable salt thereof. The combination therapy is useful for the treatment of a variety of cancers, including uveal melanoma. The combination therapy can also be useful for treatment of a solid tumor harboring GNAQ or GNA11 mutations. In another aspect, the combination therapy is useful for the treatment of any number of PKC-associated and/or c-MET-associated diseases.

Administering a combination of Compound 1 and Compound 2 can provide beneficial effects for treating cancer, e.g., uveal melanoma, in a subject. Such an approach—combination or co-administration of the two types of agents—may offer an uninterrupted treatment to an subject in need over a clinically relevant treatment period.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods. For example, a dose of about 300 mg may be understood to mean that the dose may vary between 270 mg and 330 mg.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a dose range of “200 mg to about 600 mg” should be interpreted to include not only the explicitly recited concentration of about 200 mg to about 600 mg, but also include individual dosage (e.g., 250 mg, 400 mg, 550 mg) and the sub-ranges (e.g., 250 mg to 450 mg) within the indicated range. To further illustrate, a tumor size reduction of “30%-50%” should be interpreted to include not only the explicitly recited concentration of about 30% to about 50%, but also include individual percentages (e.g., 35%, 40%, 50%) and the sub-ranges (e.g., 35%-45%) within the indicated range. The term “about” can include ±1%, ±2%, ±3%, ±4%, ±5%, ±6%, ±7%, ±8%, ±9%, or ±10%, of the numerical value(s) being modified. In addition, the phrase “about ‘x’ to ‘y’” includes “about ‘x’ to about ‘y’”.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals.

As used herein, the term “non-fixed combination” means that the active ingredients, e.g., Compound 1 and Compound 2, are both administered to a patient as separate entities either simultaneously, concurrently or sequentially, wherein such administration provides therapeutically effective levels of the two compounds in the body of the patient.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (i.e., in separate dosage units, for example, separate tablets, capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the agents are administered to the same patient as part of the same course of therapy. The agents may be administered at the same point in time or immediately following one another. The agents may be administered at any order. The agents may be administered separately at different times during the course of therapy in such time intervals that the combination therapy is effective in treating cancer. In any case, the treatment regimen will provide beneficial effects in treating the conditions or disorders described herein.

As used herein, the term “free base equivalent” refers to the amount of the active agent (e.g., Compound 1 or Compound 2) present in the active agent or pharmaceutically acceptable salt thereof. Stated alternatively, the term “free base equivalent” means either an amount of Compound 1 or Compound 2 free base, or the equivalent amount of Compound 1 or Compound 2 free base that is provided by a salt of said compound.

As used herein, “metastasis” or “metastatic” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the term “First-Line MUM patients” refers to patients that were not given prior systemic treatment in the metastatic setting including no prior embolization, no radiation to the metastatic sites or ablation to the liver lesions.

As used herein, the term “Any-Line MUM patients” refers to all patients that meet the patient eligibility criteria in the clinical trial as disclosed herein. Any-line MUM patients include (1) First-line MUM patients and (2) patients who had received prior therapies other than the combination (Compound 1 and Compound 2) as disclosed herein.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods.

Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein “RECIST 1.1” refers to Response Evaluation Criteria In Solid Tumors (RECIST) guidelines version 1.1. See. Eisenhauer et al. New response evaluation criteria in solid tumours: revised RECIST guideline (version 1.1). Eur J Cancer. 2009 January; 45 (2): 228-47.

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “unit dose” is used herein to mean simultaneous administration of both agents together, in one dosage form, to the patient being treated. In some embodiments, the unit dose is a single formulation. The term “a unit dose,” as used herein can also refer to the simultaneous administration of both agents separately, in two dosage forms, to the patient being treated. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time.

The dose amounts (for Compound 1 and Compound 2) are expressed as free base equivalent amounts, unless indicated otherwise.

The combination of agents described herein may display a synergistic effect. See, for example, Wagle, M-C., et al., Preclinical evaluation of PKC and MET inhibitor combination in primary and metastatic uveal melanoma, AACR Meeting 2021, the entire content of which is hereby incorporated by reference. The term “synergistic effect” as used herein, refers to action of two agents such as, for example, Compound 1 and Compound 2, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6:429-453 (1981)), the equation of Loewe additivity (Loewe, S. and Muischnek, H., Arch. Exp. Pathol Pharmacol. 114:313-326 (1926)) and the median-effect equation (Chou, T. C. and Talalay, P., Adv. Enzyme Regul. 22:27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., Compound 1 and Compound 2, used together is greater than the sum of the effects that results from using the compounds separately.

In an embodiment, provided herein is a combination therapy comprising an effective amount of Compound 1 and Compound 2. An “effective amount” of a combination of agents (i.e., Compound 1 and Compound 2) is an amount sufficient to provide an observable improvement over the baseline clinically observable signs and symptoms of the disorders treated with the combination.

An “oral dosage form” includes a unit dosage form prescribed or intended for oral administration.

### Combination Product

Provided herein is a combination product comprising Compound 1, or a pharmaceutically acceptable salt thereof, and Compound 2, or a pharmaceutically acceptable salt thereof. The combination product is useful for the treatment of a variety of cancers, including uveal melanoma, for example uveal melanoma harboring GNAQ or GNA11 mutations; or metastatic uveal melanoma. The combination product can also be useful for treatment of a solid tumor harboring GNAQ or GNA11 mutations. In another aspect, the combination product is useful for the treatment of any number of PKC-associated and/or c-MET associated diseases. In an aspect, the combination product is useful for the treatment of c-MET amplified tumors or tumors having high c-MET expression, for example, NSCLC, CRC (colorectal cancer), gastric cancer, HCC (hepatocellular carcinoma). In one aspect, the disclosure provides a combination which is synergistic. In some such embodiments, the disclosure provides a combination comprising: (i) Compound 1, or a pharmaceutically acceptable salt thereof; and (ii) Compound 2, or a pharmaceutically acceptable salt thereof described herein; for use in the treatment of uveal melanoma or a solid tumor harboring GNAQ or GNA11 mutations in a subject, wherein component (i) and component (ii) are synergistic.

The compound 3-amino-N-(3-(4-amino-4-methylpiperidin-1-yl) pyridin-2-yl)-6-(3-(trifluoromethyl) pyridin-2-yl) pyrazine-2-carboxamide (also known as 3-amino-N-[3-(4-amino-4-methyl-1-piperidinyl)-2-pyridinyl]-6-[3-(trifluoromethyl)-2-pyridinyl]-2-pyrazinecarboxamide), has the following structure:

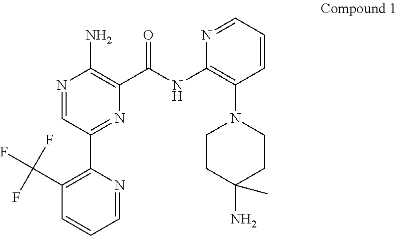 [SMILES: CC1(N)CCN(c2cccnc2NC(=O)c2nc(-c3ncccc3C(F)(F)F)cnc2N)CC1]

(which is known as darovasertib or IDE196) or a pharmaceutically acceptable salt thereof.

Compound 1 is a potent and selective inhibitor of protein kinase C. Compound 1, its synthesis, and biological activity against PKC can be found in PCT/IB2015/055951 (WO2016020864). The contents of WO2016020864 are incorporated herein by reference in their entirety.

The compound 3-[(1R)-1-(2,6-dichloro-3-fluorophenyl) ethoxy]-5-(1-piperidin-4-ylpyrazol-4-yl) pyridin-2-amine (“Compound 2”), has the following structure:

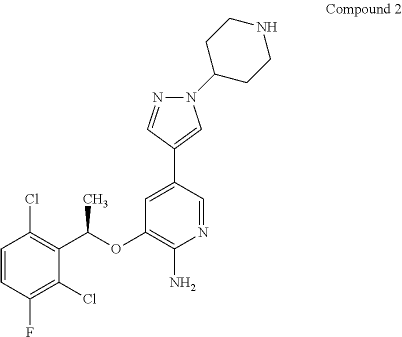 [SMILES: C[C@@H](Oc1cc(-c2cnn(C3CCNCC3)c2)cnc1N)c1c(Cl)ccc(F)c1Cl]

(which is known as crizotinib or PF-02341066) or a pharmaceutically acceptable salt thereof.

Crizotinib is an inhibitor of anaplastic lymphoma kinase (ALK) and its oncogenic variants (i.e., ALK fusion events and selected oncogenic ALK mutations), as well as the hepatocyte growth factor receptor (HGFR, c-Met), c-ros oncogene 1 (Ros1) and its oncogenic variants, and Recepteur d'Origine Nantais (RON) receptor tyrosine kinases (RTKs).

Crizotinib, as well as pharmaceutically acceptable salts thereof, is described in International Publication Nos. WO 2006/021884, WO 2006/021881 and WO 2007/066185, and in U.S. Pat. Nos. 7,858,643, 8,217,057 and 8,785,632. The use of crizotinib in treating abnormal cell growth, such as cancers, mediated by ALK or c-MET/HGFR is described in U.S. Pat. No. 7,825,137. The use of crizotinib in treating ROS mediated cancers is described in WO 2013/017989. The contents of each of the foregoing patents and applications are incorporated herein by reference in their entirety.

Thus, in an aspect, provided herein is a combination product comprising
(i) about 300 mg Compound 1:

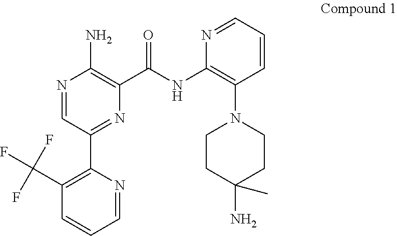 [SMILES: CC1(N)CCN(c2cccnc2NC(=O)c2nc(-c3ncccc3C(F)(F)F)cnc2N)CC1]

or an equivalent dose of a pharmaceutically acceptable salt thereof, and
(ii) about 200 mg of Compound 2:

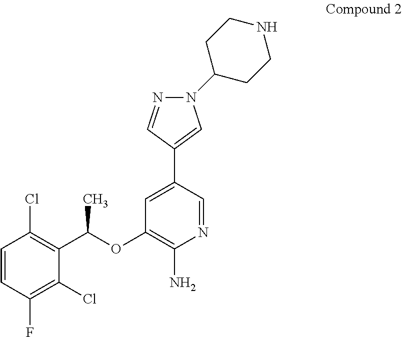 [SMILES: C[C@@H](Oc1cc(-c2cnn(C3CCNCC3)c2)cnc1N)c1c(Cl)ccc(F)c1Cl]

or an equivalent dose of a pharmaceutically acceptable salt thereof.

In another aspect, provided herein is a combination product comprising (i) about 200 mg to about 600 mg Compound 1:

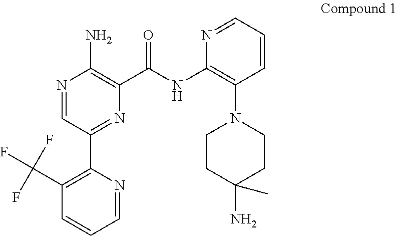 [SMILES: CC1(N)CCN(c2cccnc2NC(=O)c2nc(-c3ncccc3C(F)(F)F)cnc2N)CC1]

or an equivalent dose of a pharmaceutically acceptable salt thereof, and
(ii) about 400 mg to about 500 mg of Compound 2:

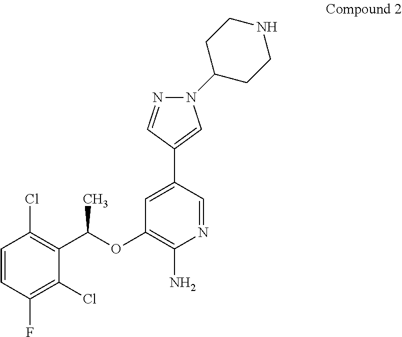 [SMILES: C[C@@H](Oc1cc(-c2cnn(C3CCNCC3)c2)cnc1N)c1c(Cl)ccc(F)c1Cl]

or an equivalent dose of a pharmaceutically acceptable salt thereof.

In yet another aspect, provided herein is a combination product comprising
(i) about 200 mg Compound 1, or an equivalent dose of a pharmaceutically acceptable salt thereof; and
(ii) about 200 mg of Compound 2, or an equivalent dose of a pharmaceutically acceptable salt thereof.

In still another aspect, provided herein is a combination product comprising
(i) about 300 mg Compound 1, or an equivalent dose of a pharmaceutically acceptable salt thereof; and
(ii) about 250 mg of Compound 2, or an equivalent dose of a pharmaceutically acceptable salt thereof.

In an embodiment, the combination product provided above comprises about 300 mg of Compound 1, or an equivalent dose of a pharmaceutically acceptable salt thereof. In another embodiment, the combination product comprises about 200 mg of Compound 2, or an equivalent dose of a pharmaceutically acceptable salt thereof.

In an embodiment, the combination product provided above comprises about 300 mg of Compound 1, or an equivalent dose of a pharmaceutically acceptable salt thereof. In another embodiment, the combination product comprises about 250 mg of Compound 2, or an equivalent dose of a pharmaceutically acceptable salt thereof.

In another embodiment, the combination product comprises Compound 1 as a free base. In yet another embodiment, the combination product comprises Compound 2 as a free base.

In an embodiment, Compound 1, or an equivalent dose of a pharmaceutically acceptable salt thereof, is present in the combination product at a dose selected from the group consisting of 200 mg, 250 mg, 300 mg, 350 mg, 400 mg, 450 mg, 500 mg, 550 mg, and 600 mg. In another embodiment, Compound 1, or an equivalent dose of a pharmaceutically acceptable salt thereof, is present in the combination product at a dose of 200 mg. In yet another embodiment, Compound 1, or an equivalent dose of a pharmaceutically acceptable salt thereof, is present in the combination product at a dose of 300 mg. In still another embodiment, Compound 1, or an equivalent dose of a pharmaceutically acceptable salt thereof, is orally administered as a tablet. In another embodiment, Compound 1, or an equivalent dose of a pharmaceutically acceptable salt thereof, is administered twice daily (BID).

In another embodiment, Compound 1, or an equivalent dose of a pharmaceutically acceptable salt thereof, is present in the combination product at a dose of 200 mg once daily (QD). In yet another embodiment, Compound 1, or an equivalent dose of a pharmaceutically acceptable salt thereof, is present in the combination product at a dose of 300 mg once daily (QD).

In an embodiment, Compound 2, or an equivalent dose of a pharmaceutically acceptable salt thereof, is present in the combination product at a dose selected from the group consisting of 200 mg, 250 mg, 300 mg, 350 mg, 400 mg, 450 mg, 500 mg, 550 mg, and 600 mg. In another embodiment, Compound 2, or an equivalent dose of a pharmaceutically acceptable salt thereof, is present in the combination product at a dose of 200 mg. In yet another embodiment, Compound 2, or an equivalent dose of a pharmaceutically acceptable salt thereof, is present in the combination product at a dose of 250 mg. In still another embodiment, Compound 2, or an equivalent dose of a pharmaceutically acceptable salt thereof, is orally administered as a tablet. In another embodiment, Compound 2, or an equivalent dose of a pharmaceutically acceptable salt thereof, is administered twice daily (BID).

In another embodiment, Compound 2, or an equivalent dose of a pharmaceutically acceptable salt thereof, is present in the combination product at a dose of 200 mg once daily (QD). In yet another embodiment, Compound 2, or an equivalent dose of a pharmaceutically acceptable salt thereof, is present in the combination product at a dose of 250 mg once daily (QD).

In an embodiment, Compound 1, or an equivalent dose of a pharmaceutically acceptable salt thereof, is present in the combination product at a dose of 200 mg BID, and Compound 2, or an equivalent dose of a pharmaceutically acceptable salt thereof, is present in the combination product at a dose of 200 mg BID. In another embodiment, Compound 1, or an equivalent dose of a pharmaceutically acceptable salt thereof, is present in the combination product at a dose of 300 mg BID, and Compound 2, or an equivalent dose of a pharmaceutically acceptable salt thereof, is present in the combination product at a dose of 200 mg BID. In yet another embodiment, Compound 1, or an equivalent dose of a pharmaceutically acceptable salt thereof, is present in the combination product at a dose of 200 mg BID, and Compound 2, or an equivalent dose of a pharmaceutically acceptable salt thereof, is present in the combination product at a dose of 250 mg BID. In yet another embodiment, Compound 1, or an equivalent dose of a pharmaceutically acceptable salt thereof, is present in the combination product at a dose of 300 mg BID, and Compound 2, or an equivalent dose of a pharmaceutically acceptable salt thereof, is present in the combination product at a dose of 250 mg BID.

In an embodiment, Compound 1, or an equivalent dose of a pharmaceutically acceptable salt thereof, is present in the combination product at a dose of 200 mg, and Compound 2, or an equivalent dose of a pharmaceutically acceptable salt thereof, is present in the combination product at a dose of 200 mg. In another embodiment, Compound 1, or an equivalent dose of a pharmaceutically acceptable salt thereof, is present in the combination product at a dose of 300 mg, and Compound 2, or an equivalent dose of a pharmaceutically acceptable salt thereof, is present in the combination product at a dose of 200 mg. In yet another embodiment, Compound 1, or an equivalent dose of a pharmaceutically acceptable salt thereof, is present in the combination product at a dose of 200 mg, and Compound 2, or an equivalent dose of a pharmaceutically acceptable salt thereof, is present in the combination product at a dose of 250 mg. In yet another embodiment, Compound 1, or an equivalent dose of a pharmaceutically acceptable salt thereof, is present in the combination product at a dose of 300 mg, and Compound 2, or an equivalent dose of a pharmaceutically acceptable salt thereof, is present in the combination product at a dose of 250 mg.

In one embodiment, any of the above-described combination products is a non-fixed combination product.

The administration of a pharmaceutical combination provided herein may result in a beneficial effect, e.g., a synergistic therapeutic effect, e.g., with regard to alleviating, delaying progression of or inhibiting the symptoms, and may also result in further surprising beneficial effects, e.g., fewer side-effects, an improved quality of life or a decreased morbidity, compared with a monotherapy applying only one of the pharmaceutically active ingredients used in the combination of the invention.

In one embodiment of the foregoing embodiments, the subject is a First-Line subject. In another embodiment of the foregoing embodiments, the subject is a First-Line MUM subject.

### Methods of Treatment

In an aspect, provided herein is a method of treating uveal melanoma or a solid tumor harboring GNAQ or GNA11 mutations in a subject in need thereof, comprising administering to the subject Compound 1:

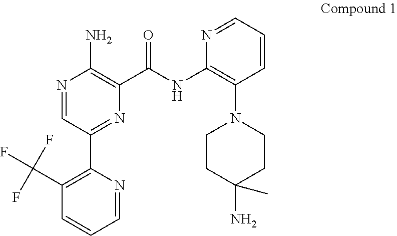 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

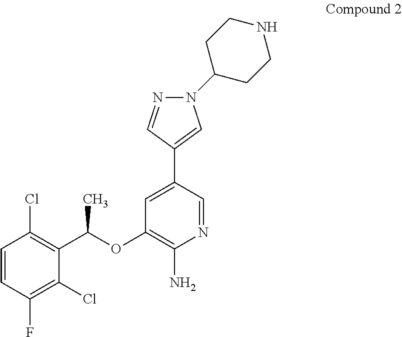 [SMILES: C[C@@H](Oc1cc(-c2cnn(C3CCNCC3)c2)cnc1N)c1c(Cl)ccc(F)c1Cl] [0116] or a pharmaceutically acceptable salt thereof;
   wherein
Compound 1, or a pharmaceutically acceptable salt thereof, is administered at a dose of about 300 mg two times per day (BID) of free base equivalent of Compound 1; and
Compound 2, or a pharmaceutically acceptable salt thereof, is administered at a dose of about 200 mg BID of free base equivalent of Compound 2.

In another aspect, provided herein is a method of treating uveal melanoma or a solid tumor harboring GNAQ or GNA11 mutations in a subject in need thereof, comprising administering to the subject Compound 1:

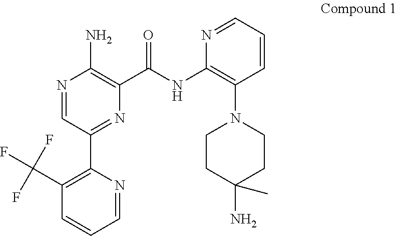 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

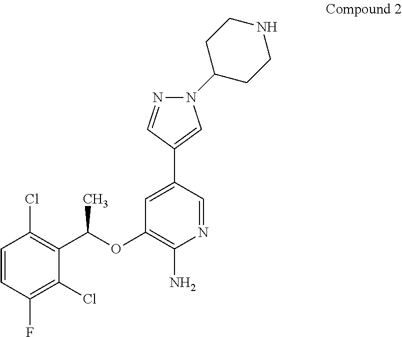 [SMILES: C[C@@H](Oc1cc(-c2cnn(C3CCNCC3)c2)cnc1N)c1c(Cl)ccc(F)c1Cl] [0122] or a pharmaceutically acceptable salt thereof;
   wherein
Compound 1, or a pharmaceutically acceptable salt thereof, is administered at a dose of about 400 mg to about 600 mg daily of free base equivalent of Compound 1; and
Compound 2, or a pharmaceutically acceptable salt thereof, is administered at a dose of about 400 mg to about 500 mg daily of free base equivalent of Compound 2.

In another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; wherein
Compound 1, or an equivalent dose of a pharmaceutically acceptable salt thereof, is administered at a dose of about 200 mg two times per day (BID); and
Compound 2, or an equivalent dose of a pharmaceutically acceptable salt thereof, is administered at a dose of about 200 mg BID.

In still another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; wherein
Compound 1, or an equivalent dose of a pharmaceutically acceptable salt thereof, is administered at a dose of about 300 mg two times per day (BID); and
Compound 2, or an equivalent dose of a pharmaceutically acceptable salt thereof, is administered at a dose of about 250 mg BID.

In another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; wherein
Compound 1, or a pharmaceutically acceptable salt thereof, is administered at a dose of about 600 mg daily of free base equivalent of Compound 1; and
Compound 2, or a pharmaceutically acceptable salt thereof, is administered at a dose of about 400 mg daily of free base equivalent of Compound 2.

In still another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;

wherein Compound 1, or a pharmaceutically acceptable salt thereof, is administered at a dose of about 300 mg of free base equivalent of Compound 1; and Compound 2, or a pharmaceutically acceptable salt thereof, is administered at a dose of about 250 mg of free base equivalent of Compound 2.

In an embodiment, the uveal melanoma is metastatic uveal melanoma. In another embodiment, the uveal melanoma is a solid tumor harboring GNAQ or GNA11 mutations. In another embodiment, the uveal melanoma is metastatic uveal melanoma harboring GNAQ or GNA11 mutations.

In yet another embodiment, the solid tumor harboring GNAQ or GNA11 mutations is cutaneous melanoma.

In still another embodiment, Compound 1, or a pharmaceutically acceptable salt thereof, is administered at a dose of about 200 mg BID of free base equivalent of Compound 1. In an embodiment, Compound 1, or a pharmaceutically acceptable salt thereof, is administered at a dose of about 300 mg BID of free base equivalent of Compound 1. In another embodiment, Compound 1, or a pharmaceutically acceptable salt thereof, is administered at a dose of about 200 mg once daily (QD) of free base equivalent of Compound 1. In yet another embodiment, Compound 1, or a pharmaceutically acceptable salt thereof, is administered at a dose of about 300 mg once daily (QD) of free base equivalent of Compound 1.

In another embodiment, Compound 2, or a pharmaceutically acceptable salt thereof, is administered at a dose of about 200 mg BID of free base equivalent of Compound 2. In an embodiment, Compound 2, or a pharmaceutically acceptable salt thereof, is administered at a dose of about 250 mg BID of free base equivalent of Compound 2. In another embodiment, Compound 2, or a pharmaceutically acceptable salt thereof, is administered at a dose of about 200 mg once daily (QD) of free base equivalent of Compound 2. In yet another embodiment, Compound 2, or a pharmaceutically acceptable salt thereof, is administered at a dose of about 250 mg once daily (QD) of free base equivalent of Compound 2.

In another embodiment, the administration of Compound 1, or a pharmaceutically acceptable salt thereof, is uninterrupted during a dosing schedule comprising at least one 7-day dosing cycle.

In yet another embodiment, Compound 1, or a pharmaceutically acceptable salt thereof, and Compound 2, or a pharmaceutically acceptable salt thereof, are administered in a single formulation. In still another embodiment, Compound 1, or a pharmaceutically acceptable salt thereof, and Compound 2, or a pharmaceutically acceptable salt thereof, are administered in a single formulation further comprising one or more pharmaceutically acceptable carriers.

In an embodiment, Compound 1, or a pharmaceutically acceptable salt thereof, and Compound 2, or a pharmaceutically acceptable salt thereof, are administered separately.

In another embodiment, the treatment comprises administering Compound 1, or a pharmaceutically acceptable salt thereof, and Compound 2, or a pharmaceutically acceptable salt thereof, at substantially the same time. In yet another embodiment, the treatment comprises administering Compound 1, or a pharmaceutically acceptable salt thereof, and Compound 2, or a pharmaceutically acceptable salt thereof, at different times.

In still another embodiment, Compound 1, or a pharmaceutically acceptable salt thereof, is administered to the subject, followed by administration of Compound 2, or a pharmaceutically acceptable salt thereof. In an embodiment, Compound 2, or a pharmaceutically acceptable salt thereof, is administered to the subject, followed by administration of Compound 1, or a pharmaceutically acceptable salt thereof.

In an embodiment, administering Compound 1, or a pharmaceutically acceptable salt thereof, and Compound 2, or a pharmaceutically acceptable salt thereof, reduces the size of one or more lesions of the uveal melanoma or the solid tumor harboring GNAQ or GNA11 mutations in said subject.

In another embodiment, administering Compound 1, or a pharmaceutically acceptable salt thereof, and Compound 2, or a pharmaceutically acceptable salt thereof, decreases the growth rate of one of more lesions of the uveal melanoma or the solid tumor harboring GNAQ or GNA11 mutations in said subject.

In yet another embodiment, the method comprises administering to the subject in need thereof Compound 1:

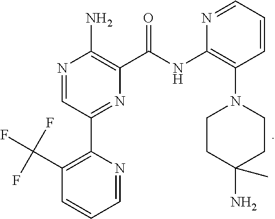 [SMILES: CC1(N)CCN(c2cccnc2NC(=O)c2nc(-c3ncccc3C(F)(F)F)cnc2N)CC1]

In still another embodiment, the method comprises administering to the subject in need thereof Compound 2:

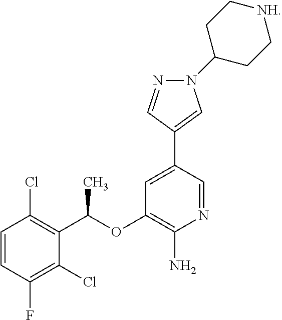 [SMILES: C[C@@H](Oc1cc(-c2cnn(C3CCNCC3)c2)cnc1N)c1c(Cl)ccc(F)c1Cl]

In an embodiment, Compound 1, or a pharmaceutically acceptable salt thereof, and Compound 2, or a pharmaceutically acceptable salt thereof, are administered orally.

In an embodiment, an uninterrupted treatment cycle indicates that the subject or patient does not miss a dose of Compound 1 and/or Compound 2. For example, if Compound 1 is administered uninterrupted at 300 mg BID for one 7-day dosing cycle, 300 mg of Compound 1 is administered two times per day for 7 consecutive days.

In another embodiment, the method of treating uveal melanoma or a solid tumor harboring GNAQ or GNA11 mutations in a subject in need thereof can further comprise: (i) first administering Compound 1, or a pharmaceutically acceptable salt thereof, and Compound 2, or a pharmaceutically acceptable salt thereof, to the patient in need thereof according to the dosing regimen of any of the aforementioned embodiments over a treatment period comprising at least 4 consecutive 7-day dosing cycles, then (ii) increasing the dose of Compound 1, or a pharmaceutically acceptable salt thereof, to 300 mg BID or 600 mg total daily as applicable, and/or (iii) lowering the dose of Compound 2, or a pharmaceutically acceptable salt thereof, to 200 mg BID or 250 mg total daily as applicable.

In yet another embodiment, the method of treating uveal melanoma or a solid tumor harboring GNAQ or GNA11 mutations in a subject in need thereof can further comprise: continuing to administer Compound 1, or a pharmaceutically acceptable salt thereof, and Compound 2, or a pharmaceutically acceptable salt thereof, uninterrupted during a dosing schedule comprising at least two or more consecutive 7-day dosing cycles, or preferably at least four or more consecutive 7-day dosing cycles. In particular embodiments, Compound 1, or a pharmaceutically acceptable salt thereof, and Compound 2, or a pharmaceutically acceptable salt thereof, are continued to be co-administered uninterrupted during a dosing schedule comprising at least eight or more, twelve or more, twenty-four or more, forty-eight or more or ninety-six or more consecutive 7-day dosing cycles. In an embodiment, Compound 1, or a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles. In another embodiment, Compound 2, or a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles.

Exemplary lengths of time associated with the course of the treatment methods is about five years, about, 4 years, about 3 years, about 2 years, about 1 years, about 11 months, about 10 months, about 9 months, about 8 months, about 7 months, about 6 months, about 5 months, about 4 months, about 3 months, about 2 months, or about 1 month.

Exemplary lengths of time associated with the course of the treatment methods is about five years and so on; or any days, weeks, months, or years in between; for example a treatment cycle can include 5 months and additional weeks and/or days, or one year and additional months, weeks, and/or days, and the like.

In some embodiments, Compound 1, or a pharmaceutically acceptable salt thereof, and Compound 2, or a pharmaceutically acceptable salt thereof, are administered continuously (i.e., a continuous treatment until termination).

The GNAQ or GNA11 tumor to be treated can include one or more of a number of mutations, including a substitution mutation, an insertion mutation, and/or a deletion in GNAQ or GNA11 mutation. In some aspects, the GNAQ or GNA11 mutation is a gain of function mutation. In some aspects, the GNAQ or GNA11 mutation activates the PKC signaling pathway. In various aspects, the GNAQ or GNA11 mutation can be the substitution of glutamine in codon 209 (Q209) and/or a substitution of arginine in codon 183 (R183). The GNAQ or GNA11 mutation can be a substitution other than glutamine in codon 209 (Q209), other than a substitution of arginine in codon 183 (R183), or other than both. In some aspects, the GNAQ mutation is one of Q209P, Q209L, Q209H, Q209K, or Q209Y, or the GNA11 mutation is one of Q209P, Q209L, Q209K or Q209H. In further aspects, the GNAQ mutation can be R183Q, or the GNA11 mutation can be R183C or R183H. In yet further examples, the GNAQ or GNA11 mutation is at one or more of R256, L279, R166, A168, R210, R213, R166, A231, A342, D333, G171, R147, R73, T47, E191, E221, R149, T175, T379, T85, A86, E163, D195, E319, E191, E280, E49, P293, R300, R338, R60, D155, D205, D321, 1226, R37, or V240. In further examples, the GNAQ or GNA11 tumor can comprise one or more of a Q209P, Q209L, Q209H, Q209K, Q209Y, or R183Q mutation in GNAQ, or the GNAQ or GNA11 tumor can comprise one or more of a Q209P, Q209L, Q209H, or Q209K mutation in GNA11. Additional examples of mutations in GNAQ or GNA11 are described in WO 2020/146355, which is incorporated by reference herewith in its entirety.

In another aspect, provided herein is a method of treating cancer in a subject in need thereof, comprising administering to the subject Compound 1:

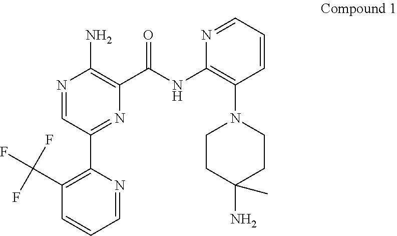 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

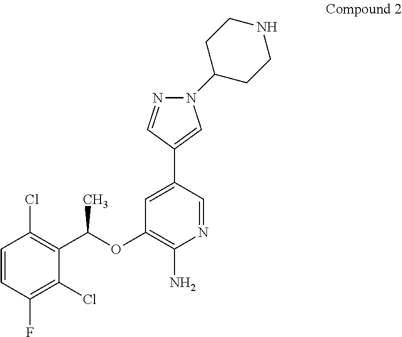 [SMILES: C[C@@H](Oc1cc(-c2cnn(C3CCNCC3)c2)cnc1N)c1c(Cl)ccc(F)c1Cl] [0160] or a pharmaceutically acceptable salt thereof;
   wherein
Compound 1, or a pharmaceutically acceptable salt thereof, is administered at a dose of about 400 mg to about 600 mg daily of free base equivalent of Compound 1; and
Compound 2, or a pharmaceutically acceptable salt thereof, is administered at a dose of about 400 mg to about 500 mg daily of free base equivalent of Compound 2.

In another aspect, provided herein is a method of treating cancer in a subject in need thereof, comprising administering to the subject Compound 1, or a pharmaceutically acceptable salt thereof, and Compound 2, or a pharmaceutically acceptable salt thereof; wherein
Compound 1, or an equivalent dose of a pharmaceutically acceptable salt thereof, is administered at a dose of about 200 mg two times per day (BID); and
Compound 2, or an equivalent dose of a pharmaceutically acceptable salt thereof, is administered at a dose of about 200 mg BID.

In yet another aspect, provided herein is a method of treating cancer in a subject in need thereof, comprising administering to the subject Compound 1, or a pharmaceutically acceptable salt thereof, and Compound 2, or a pharmaceutically acceptable salt thereof;

wherein
Compound 1, or an equivalent dose of a pharmaceutically acceptable salt thereof, is administered at a dose of about 300 mg two times per day (BID); and
Compound 2, or an equivalent dose of a pharmaceutically acceptable salt thereof, is administered at a dose of about 250 mg BID.

In an embodiment, Compound 1, or a pharmaceutically acceptable salt thereof, is administered at a dose of about 200 mg BID of free base equivalent of Compound 1, and Compound 2, or a pharmaceutically acceptable salt thereof, is administered at a dose of about 200 mg BID of free base equivalent of Compound 2. In an embodiment, Compound 1, or a pharmaceutically acceptable salt thereof, is administered at a dose of about 300 mg BID of free base equivalent of Compound 1, and Compound 2, or a pharmaceutically acceptable salt thereof, is administered at a dose of about 200 mg BID of free base equivalent of Compound 2. In an embodiment, Compound 1, or a pharmaceutically acceptable salt thereof, is administered at a dose of about 300 mg BID of free base equivalent of Compound 1, and Compound 2, or a pharmaceutically acceptable salt thereof, is administered at a dose of about 250 mg BID of free base equivalent of Compound 2.

In another embodiment, the cancer to be treated is selected from the group consisting of lung cancer, colon and rectal cancer, breast cancer, prostate cancer, liver cancer, pancreatic cancer, brain cancer, kidney cancer, ovarian cancer, stomach cancer, skin cancer, bone cancer, gastric cancer, glioma, glioblastoma, neuroblastoma, hepatocellular carcinoma, papillary renal carcinoma, head and neck squamous cell carcinoma, leukemia, lymphomas, myelomas, retinoblastoma, cervical cancer, melanoma and/or skin cancer, bladder cancer, uterine cancer, testicular cancer, esophageal cancer, and solid tumors. In some embodiments, the cancer is lung cancer, colon cancer, breast cancer, neuroblastoma, leukemia, and lymphomas. In other embodiments, the cancer is lung cancer, colon cancer, breast cancer, neuroblastoma, leukemia, or lymphoma. In a further embodiment, the cancer is non-small cell lung cancer (NSCLC) or small cell lung cancer. In a further embodiment, the cancer is hepatocellular carcinoma (HCC). In a further embodiment, the cancer is gastric cancer. In a further embodiment, the cancer is colorectal cancer. In another embodiment, the cancer is parotid gland carcinoma.

In another embodiment, the cancer to be treated is a solid tumor harboring GNA11 or GNAQ mutations. In yet another embodiment, the cancer is uveal melanoma. In yet another embodiment, the cancer is uveal melanoma harboring GNA11 or GNAQ mutations. In still another embodiment, the cancer is metastatic uveal melanoma. In still another embodiment, the cancer is metastatic uveal melanoma harboring GNA11 or GNAQ mutations. In an embodiment, the cancer is cutaneous melanoma. In an embodiment, the cancer is cutaneous melanoma harboring GNA11 or GNAQ mutations. In an embodiment, the cancer is parotid gland carcinoma harboring GNA11 or GNAQ mutations.

In another embodiment, the cancer to be treated is a tumor having c-MET amplification or high expression of c-MET. In another embodiment, the cancer to be treated is a tumor having high expression of c-MET. The c-MET amplification can be determined using various techniques for example, next generation sequencing (NGS) or fluorescence in situ hybridization (FISH). High c-MET expression can be determined using techniques such as immunohistochemistry (IHC). In an embodiment, the c-MET amplified cancer is NSCLC or HCC. In another embodiment, the cancer having high c-MET expression are NSCLC, HCC, colorectal cancer, or gastric cancer.

In an embodiment, the cancer is a hematologic cancer, such as leukemia or lymphoma. In a certain embodiment, lymphoma is Hodgkin's lymphoma or Non-Hodgkin's lymphoma. In certain embodiments, leukemia is myeloid, lymphocytic, myelocytic, lymphoblastic, or megakaryotic leukemia.

In an embodiment, provided herein is Compound 1, or a pharmaceutically acceptable salt thereof, and Compound 2, or a pharmaceutically acceptable salt thereof, for use in therapy.

In an embodiment, provided herein is Compound 1, or a pharmaceutically acceptable salt thereof, and Compound 2, or a pharmaceutically acceptable salt thereof, for use in treating uveal melanoma in a patient in need thereof.

In an embodiment, provided herein is Compound 1, or a pharmaceutically acceptable salt thereof, and Compound 2, or a pharmaceutically acceptable salt thereof, for use in treating metastatic uveal melanoma in a patient in need thereof.

In an embodiment, provided herein is Compound 1, or a pharmaceutically acceptable salt thereof, and Compound 2, or a pharmaceutically acceptable salt thereof, for use in treating solid tumors harboring GNAQ or GNA11 mutations in a patient in need thereof.

In another embodiment, provided herein is Compound 1 and Compound 2 for use in treating cutaneous melanoma in a patient in need thereof.

In an embodiment, provided herein is Compound 1, or a pharmaceutically acceptable salt thereof, and Compound 2, or a pharmaceutically acceptable salt thereof, for the preparation of a medicament for treating or otherwise alleviating a symptom of a PKC-associated and/or c-MET-associated diseases as described herein, such as uveal melanoma or a tumor having a mutation in GNAQ or GNA11. In an embodiment, the uveal melanoma is metastatic uveal melanoma. In another embodiment, the uveal melanoma is a solid tumor harboring GNAQ or GNA11 mutations. In another embodiment, the uveal melanoma is metastatic uveal melanoma harboring GNAQ or GNA11 mutations. In an embodiment, the disease is a tumor having a mutation in GNAQ or GNA11.

Exemplary lengths of time associated with the course of the treatment methods disclosed herein include: about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about 4 months; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years and so on; or any days, weeks, months, or years in between; for example a treatment cycle can include 5 months and additional weeks and/or days, or one year and additional months, weeks, and/or days, and the like.

Exemplary lengths of time associated with the course of the treatment methods can be about five years, about 4 years, about 3 years, about 2 years, about 1 years, about 11 months, about 10 months, about 9 months, about 8 months, about 7 months, about 6 months, about 5 months, about 4 months, about 3 months, about 2 months, or about 1 month.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of Compound 1, or a pharmaceutically acceptable salt thereof, and the amount of Compound 2, or a pharmaceutically acceptable salt thereof. In an embodiment, the amount of Compound 1, or a pharmaceutically acceptable salt thereof, and the amount of Compound 2, or a pharmaceutically acceptable salt thereof, are in a single formulation or unit dosage form. In still other embodiments, the amount of Compound 1, or a pharmaceutically acceptable salt thereof, and the amount of Compound 2, or a pharmaceutically acceptable salt thereof, are in a separate formulations or unit dosage forms.

In the foregoing methods, the treatment can include administering the amount of Compound 1, or a pharmaceutically acceptable salt thereof, and the amount of Compound 2, or a pharmaceutically acceptable salt thereof, at substantially the same time or administering the amount of Compound 1, or a pharmaceutically acceptable salt thereof, and the amount of Compound 2, or a pharmaceutically acceptable salt thereof, at different times. In some embodiments of the foregoing methods, the amount of Compound 1, or a pharmaceutically acceptable salt thereof, and/or the amount of Compound 2, or a pharmaceutically acceptable salt thereof, is administered at dosages that would not be effective when one or both of Compound 1, or a pharmaceutically acceptable salt thereof, and Compound 2, or a pharmaceutically acceptable salt thereof, is administered alone, but which amounts are effective in combination.

In one embodiment of the foregoing methods, the treatment reduces the size of one of more lesions of the uveal melanoma or the solid tumor harboring GNAQ or GNA11 mutations in said subject.

In one embodiment of the foregoing methods, the treatment reduces the size of one of more lesions of the metastatic uveal melanoma in said subject.

In one embodiment of the foregoing methods, the treatment reduces the size of one of more lesions of the solid tumor harboring GNAQ or GNA11 mutations in said subject.

In one embodiment of the foregoing methods, the treatment decreases the growth rate of one of more lesions of the uveal melanoma or the solid tumor harboring GNAQ or GNA11 mutations in said subject.

In one embodiment of the foregoing methods, the treatment decreases the growth rate of one of more lesions of the metastatic uveal melanoma in said subject.

In one embodiment of the foregoing methods, the treatment decreases the growth rate of one of more lesions of the solid tumor harboring GNAQ or GNA11 mutations in said subject.

In one embodiment of the foregoing methods, the subject is a First-Line subject. In another embodiment, the subject is a First-Line MUM subject.

### Pharmaceutical Compositions

In an aspect, provided herein is a pharmaceutical composition comprising Compound 1, or a pharmaceutically acceptable salt thereof, Compound 2, or a pharmaceutically acceptable salt thereof, and at least one pharmaceutically acceptable carrier. In an embodiment, the pharmaceutical composition is for use in the treatment of uveal melanoma in a patient. In yet another embodiment, the uveal melanoma is a solid tumor harboring GNAQ or GNA11 mutations. In yet another embodiment, the uveal melanoma is metastatic uveal melanoma. In yet another embodiment, the metastatic uveal melanoma is a solid tumor harboring GNAQ or GNA11 mutations. In an embodiment, the pharmaceutical composition is for use in the treatment of a solid tumor harboring GNAQ or GNA11 mutations in a patient. In an embodiment, the pharmaceutical composition is for use in the treatment of a solid tumor harboring GNAQ or GNA11 mutations in a patient, wherein the solid tumor is cutaneous melanoma.

In an embodiment, the pharmaceutical composition reduces the size of one or more lesions of the uveal melanoma or the solid tumor harboring GNAQ or GNA11 mutations in said subject. In another embodiment, the pharmaceutical composition decreases the growth rate of one of more lesions of the uveal melanoma or the solid tumor harboring GNAQ or GNA11 mutations in said subject. In yet another embodiment, the uveal melanoma is metastatic uveal melanoma.

In an embodiment, Compound 1, or an equivalent dose of a pharmaceutically acceptable salt thereof, is present in the pharmaceutical composition at a dose selected from the group consisting of about 200 mg, 250 mg, 300 mg, 350 mg, 400 mg, 450 mg, 500 mg, 550 mg, and 600 mg. In another embodiment, Compound 1, or an equivalent dose of a pharmaceutically acceptable salt thereof, is present in the pharmaceutical composition at a dose of about 200 mg. In yet another embodiment, Compound 1, or an equivalent dose of a pharmaceutically acceptable salt thereof, is present in the pharmaceutical composition at a dose of about 300 mg. In yet another embodiment, Compound 1, or an equivalent dose of a pharmaceutically acceptable salt thereof, is present in the pharmaceutical composition at a dose of 300 mg. In still another embodiment, Compound 1, or an equivalent dose of a pharmaceutically acceptable salt thereof, is orally administered as a tablet. In another embodiment, Compound 1, or an equivalent dose of a pharmaceutically acceptable salt thereof, is administered twice daily (BID).

In an embodiment, Compound 2, or an equivalent dose of a pharmaceutically acceptable salt thereof, is present in the pharmaceutical composition at a dose selected from the group consisting of about 200 mg, 250 mg, 300 mg, 350 mg, 400 mg, 450 mg, 500 mg, 550 mg, and 600 mg. In another embodiment, Compound 2, or an equivalent dose of a pharmaceutically acceptable salt thereof, is present in the pharmaceutical composition at a dose of about 200 mg. In another embodiment, Compound 2, or an equivalent dose of a pharmaceutically acceptable salt thereof, is present in the pharmaceutical composition at a dose of 200 mg. In yet another embodiment, Compound 2, or an equivalent dose of a pharmaceutically acceptable salt thereof, is present in the pharmaceutical composition at a dose of about 250 mg. In still another embodiment, Compound 2, or an equivalent dose of a pharmaceutically acceptable salt thereof, is orally administered as a tablet. In another embodiment, Compound 2, or an equivalent dose of a pharmaceutically acceptable salt thereof, is administered twice daily (BID).

In an embodiment, Compound 1, or an equivalent dose of a pharmaceutically acceptable salt thereof, is present in the pharmaceutical composition at a dose of about 200 mg BID, and Compound 2, or an equivalent dose of a pharmaceutically acceptable salt thereof, is administered at a dose of about 200 mg BID. In yet another embodiment, Compound 1, or an equivalent dose of a pharmaceutically acceptable salt thereof, is present in the pharmaceutical composition at a dose of about 200 mg BID, and Compound 2, or an equivalent dose of a pharmaceutically acceptable salt thereof, is present in the pharmaceutical composition at a dose of about 250 mg BID. In another embodiment, Compound 1, or an equivalent dose of a pharmaceutically acceptable salt thereof, is present in the pharmaceutical composition at a dose of about 300 mg BID, and Compound 2, or an equivalent dose of a pharmaceutically acceptable salt thereof, is administered at a dose of about 200 mg BID. In yet another embodiment, Compound 1, or an equivalent dose of a pharmaceutically acceptable salt thereof, is present in the pharmaceutical composition at a dose of about 300 mg BID, and Compound 2, or an equivalent dose of a pharmaceutically acceptable salt thereof, is present in the pharmaceutical composition at a dose of about 250 mg BID.

In an embodiment, Compound 1, or a pharmaceutically acceptable salt thereof, is present in the pharmaceutical composition at a dose of 300 mg BID of free base equivalent of Compound 1, and Compound 2, or a pharmaceutically acceptable salt thereof, is administered at a dose of 200 mg BID of free base equivalent of Compound 2.

In an embodiment, Compound 1, or a pharmaceutically acceptable salt thereof, is present in the pharmaceutical composition at a dose of 200 mg BID of free base equivalent of Compound 1, and Compound 2, or a pharmaceutically acceptable salt thereof, is administered at a dose of 200 mg BID of free base equivalent of Compound 2.

In an embodiment, Compound 1, or a pharmaceutically acceptable salt thereof, is present in the pharmaceutical composition at a dose of 300 mg BID of free base equivalent of Compound 1, and Compound 2, or a pharmaceutically acceptable salt thereof, is administered at a dose of 250 mg BID of free base equivalent of Compound 2.

In an embodiment, Compound 1, or a pharmaceutically acceptable salt thereof, is present in the pharmaceutical composition at a dose of about 200 mg of free base equivalent of Compound 1, and Compound 2, or a pharmaceutically acceptable salt thereof, is administered at a dose of about 200 mg of free base equivalent of Compound 2. In yet another embodiment, Compound 1, or a pharmaceutically acceptable salt thereof, is present in the pharmaceutical composition at a dose of about 200 mg of free base equivalent of Compound 1, and Compound 2, or a pharmaceutically acceptable salt thereof, is present in the pharmaceutical composition at a dose of about 250 mg of free base equivalent of Compound 2. In another embodiment, Compound 1, or a pharmaceutically acceptable salt thereof, is present in the pharmaceutical composition at a dose of about 300 mg of free base equivalent of Compound 2, and Compound 2, or a pharmaceutically acceptable salt thereof, is administered at a dose of about 200 mg of free base equivalent of Compound 2. In yet another embodiment, Compound 1, or a pharmaceutically acceptable salt thereof, is present in the pharmaceutical composition at a dose of about 300 mg of free base equivalent of Compound 1, and Compound 2, or a pharmaceutically acceptable salt thereof, is present in the pharmaceutical composition at a dose of about 250 mg of free base equivalent of Compound 2.

In another aspect, provided herein is a pharmaceutical composition or pharmaceutical combination comprising the compounds disclosed herein, together with a pharmaceutically acceptable carrier.

In another aspect, provided herein is a pharmaceutical composition comprising Compound 1, or a pharmaceutically acceptable salt thereof, and Compound 2, or a pharmaceutically acceptable salt thereof. In another embodiment, the pharmaceutical composition of Compound 1, or a pharmaceutically acceptable salt thereof, and Compound 2, or a pharmaceutically acceptable salt thereof, further comprises one or more pharmaceutically acceptable carriers.

In an embodiment of the pharmaceutical compositions, Compound 1, or a pharmaceutically acceptable salt thereof, and Compound 2, or a pharmaceutically acceptable salt thereof, are in the same formulation. In another embodiment of the combination product, Compound 1 and Compound 2, are in separate formulations. In a further embodiment of this embodiment, the formulations are for simultaneous or sequential administration.

Administration of the pharmaceutical composition includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

In one embodiment of the foregoing embodiments, the subject is a First-Line subject. In another embodiment of the foregoing embodiments, the subject is a First-Line MUM subject.

### Administration/Dosage/Formulations

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating uveal melanoma or a solid tumor harboring GNAQ or GNA11 mutations, comprising Compound 1, or an equivalent dose of a pharmaceutically acceptable salt thereof, in a unit dosage of about 400 mg to about 600 mg, and Compound 2, or an equivalent dose of a pharmaceutically acceptable salt thereof, in a unit dosage of about 400 mg to about 500 mg. In another embodiment, the present disclosure provides a kit for treating uveal melanoma or a solid tumor harboring GNAQ or GNA11 mutations, comprising Compound 1, or an equivalent dose of a pharmaceutically acceptable salt thereof, in an amount of about 300 mg, and Compound 2, or an equivalent dose of a pharmaceutically acceptable salt thereof, in an amount of about 200 mg. In another embodiment, the present disclosure provides a kit for treating uveal melanoma or a solid tumor harboring GNAQ or GNA11 mutations, comprising Compound 1, or an equivalent dose of a pharmaceutically acceptable salt thereof, in an amount from about 400 mg per day to about 600 mg per day, and Compound 2, or an equivalent dose of a pharmaceutically acceptable salt thereof, in an amount from about 400 mg per day to about 500 mg per day. In some embodiments, the kit further comprises packaging and instructions. In yet another embodiment, the uveal melanoma is metastatic uveal melanoma. In yet another embodiment, the metastatic uveal melanoma is a solid tumor harboring GNAQ or GNA11 mutations. In an embodiment, the kit is for use in the treatment of a solid tumor harboring GNAQ or GNA11 mutations in a patient. In an embodiment, the kit is for use in the treatment of a solid tumor harboring GNAQ or GNA11 mutations in a patient, wherein the solid tumor is cutaneous melanoma.

In certain embodiments, the kit comprises a pharmaceutical product comprising a pharmaceutical composition comprising Compound 1, or a pharmaceutically acceptable salt thereof, and a pharmaceutically acceptable carrier or diluent; and a pharmaceutical composition comprising Compound 2, or a pharmaceutically acceptable salt thereof, and a pharmaceutically acceptable carrier or diluent.

In some embodiments, the kit comprises a pharmaceutical composition comprising Compound 1, or a pharmaceutically acceptable salt thereof; Compound 2, or a pharmaceutically acceptable salt thereof; and a pharmaceutically acceptable carrier or diluent.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g., an airless bottle. In other embodiments, the sealed container is a sealed tube. An instruction for the use of the composition and the information about the composition are to be included in the kit.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment, the kit contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The kits provided herein comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

Processes for preparing the compounds disclosed herein can be found, at least, in WO 2016/020864 and WO 2006/021884, the contents of which are incorporated in their entirety.

### Example 1: Clinical Data

Patients having metastatic uveal melanoma (MUM) were treated in a human clinical trial evaluating Compound 1 (darovasertib) and Compound 2 (crizotinib) in metastatic uveal melanoma (MUM), the results of which are shown in Tables 1-4.

TABLE 1

| | | | Tumor size from |
|---|---|---|---|
| | | | baseline (determined |
| | Dose: Compound 1 | | from post baseline |
| Patient | (mg)/Compound 2 | Mutation | scan) (Best of % |
| ID | (mg) | status | Change RECIST 1.1) |
| 1 | 300/200 | # | −5.4% |
| 2 | 300/200 | # | −7.1% |
| 3* | 300/200 | # | −7.5% |
| 4 | 300/200 | GNAQ(Q209P) | −9.4% |
| 5 | 300/200 | # | −11.6% |
| 6* | 300/200 | # | −12.8% |
| 7 | 300/200 | GNAQ(Q209P) | −16.7% |
| 8 | 300/200 | # | −20.0% |
| 9* | 300/200 | # | −23.3% |
| 10 | 300/200 | # | −23.7% |
| 11⁺⁺ | 300/200 | # | −27.6% |
| 12 | 300/200 | GNA11(Q209L) | −31.3% |
| 13⁺ | 300/200 | # | −31.3% |
| 14⁺ | 300/200 | # | −36.5% |
| 15⁺ | 300/200 | # | −40.4% |
| 16⁺ | 300/200 | GNAQ(Q209P) | −57.6% |

⁺Confirmed partial response by investigator or central review
⁺⁺Unconfirmed partial response by central review awaiting follow-up scans
*Has had only 1 post baseline scan
# mutational status not determined

Clinical data observation (Table 1)
1. All patients show tumor reduction in target lesions (n=16 evaluable patients with ≥1 scan)
2. Patient population is heavily pre-treated (e.g., prior therapies including 2 or more therapies) metastatic uveal melanoma (MUM) patients with ≥2 scans (n=13)*
   a. 4 patients with confirmed partial response (PRs). 3 of these 4 patients had undergone immunotherapy previously.
   b. 5 patients demonstrate >30% tumor reduction

TABLE 2

| | | | Tumor size from |
|---|---|---|---|
| | | | baseline (determined |
| | Dose: Compound 1 | | from post baseline |
| Patient | (mg)/Compound 2 | Mutation | scan) (Best of % |
| ID | (mg) | status | Change RECIST 1.1) |
| 36 | 200/200 | # | 14.3% |
| 37 | 200/200 | # | 4.0% |
| 38 | 200/200 | # | 0% |
| 39 | 200/200 | # | −27.6% |
| 40 | 200/200 | # | −76.7% |

TABLE 3

| | | | Tumor size from |
|---|---|---|---|
| | | | baseline (determined |
| | Dose: Compound 1 | | from post baseline |
| Patient | (mg)/Compound 2 | Mutation | scan) (Best of % |
| ID | (mg) | status | Change RECIST 1.1) |
| 41 | 300/250 | # | −13.0% |

Patient data (Table 2 and Table 3) based on ≥1 post baseline scan

TABLE 4

| | | | Tumor size from |
|---|---|---|---|
| | Dose: Compound 1 | | baseline (determined |
| | (mg) BID/ | | from post baseline |
| Patient | Compound 2 | Mutation | scan) (Best of % |
| ID | (mg) BID | Status | Change RECIST 1.1) |
| 1 | 300/200 | # | −5.4% |
| 2 | 300/200 | # | −7.1% |
| 3 | 300/200 | GNAQ(Q209L) | −7.5% |
| 4 | 300/200 | GNAQ(Q209P) | −9.4% |
| 5 | 300/200 | GNA11(Q209L) | −25.6% |
| 6 | 300/200 | # | −14.4% |
| 7 | 300/200 | GNAQ(Q209P) | −16.7% |
| 8* | 300/200 | GNA11(Q209L) | −48.6% |
| 9 | 300/200 | GNA11 (Q209L) | −28.8% |
| 10 | 300/200 | # | −23.7% |
| 11 | 300/200 | # | −41.4% |
| 12 | 300/200 | GNA11(Q209L) | −31.2% |
| 13 | 300/200 | GNA11(Q209P) | −33.3% |
| 14 | 300/200 | GNA11(Q209L) | −42.7% |
| 15 | 300/200 | # | −40.4% |
| 16 | 300/200 | GNAQ(Q209P) | −57.6% |
| 17 | 300/200 | # | −34.2% |
| 18 | 300/200 | # | −40.9% |
| 19 | 300/200 | GNAQ(Q209L) | −12.3% |
| 20* | 300/200 | # | −44.4% |
| 21 | 300/200 | GNAQ(Q209L) | −20.0% |
| 22* | 300/200 | # | 0.0% |
| 23 | 300/200 | # | −36.4% |
| 24* | 300/200 | # | −35.9% |
| 25 | 300/200 | GNAQ (Q209H) | −21.8% |
| 26* | 300/200 | GNAQ(Q209Y) | −25.5% |
| 27 | 300/200 | GNAQ (Q209L) | −19.4% |
| 28 | 300/200 | # | −33.3% |
| 29* | 300/200 | GNAQ(Q209L) | −55.6% |
| 30* | 300/200 | # | 51.1% |
| 31 | 300/200 | # | 38.5% |
| 32 | 300/200 | # | −13.9% |
| 33* | 300/200 | # | −35.1% |
| 34 | 300/200 | # | 0.00% |
| 35 | 300/200 | # | −1.6% |

*First-Line MUM patients

### Clinical Data Observation (Table 4)

Tumor shrinkage was observed in 89% of evaluable Any-Line MUM patients (n=31/35). Overall response rate (ORR) of 31% was observed in evaluable Any-Line MUM patients (n=11/35, Confirmed Partial Responses (cPRs)). Greater than 30% tumor shrinkage was observed in 43% evaluable Any-Line MUM patients (n=15/35). Disease control rate (DCR, including cPR, uPR (unconfirmed partial response), or SD (stable disease) as best overall response) was observed in 83% evaluable Any-Line MUM patients (n=29/35). Confirmed partial response (cPR) of 31% was observed in evaluable Any-Line MUM patients (n=11/35). Overall response rate (ORR) of 50% was observed in evaluable First-Line MUM patients (n=4/8 cPRs). Tumor shrinkage was observed in 75% of evaluable First-Line MUM patients (n=6/8). Greater than 30% tumor shrinkage was observed in 63% evaluable First-Line MUM patients (n=5/8). Disease control rate (DCR, including cPR, uPR, or SD as best overall response) was observed in 75% evaluable First-Line MUM patients (n=6/8). Confirmed partial response (cPR) of 50% was observed in evaluable First-Line MUM patients (n=6/8)

Median progression free survival (mPFS) is not yet reached but mPFS is >5 months in First-Line MUM patients and about 5 months in Any-Line MUM patients.

Median duration of response (DOR) is not yet reached in First-Line MUM patients or Any-Line patients. 7 of 11 patients with cPRs in Any-Line MUM patients remain in response. 4 of 4 patients with cPRs in First-Line MUM patients remain in response. The Table 4 data is as of Jun. 26, 2022.

### Example 2

### Patient Eligibility Criteria

### Inclusion Criteria:

Patient must be ≥18 years of age
Diagnosis of one of the following:
   MUM: Uveal melanoma with histological or cytological confirmed metastatic disease. Or
   Non-MUM: Advanced cutaneous melanoma, colorectal cancer, or other solid tumor that has progressed following prior standard therapies or that has no satisfactory alternative therapies and has evidence of GNAQ/11 hotspot mutation
Measurable disease
Eastern Cooperative Oncology Group $1 and expected life expectancy of >3 months
Adequate organ function at screening
Adequate contraceptive measures for non-sterilized male and female patients of childbearing potential.
Prior chemotherapy other therapies as applicable or major surgeries must have been completed at least 4 weeks prior to initiation of crizotinib
Patients with preexisting peripheral neuropathy can be included if it is Grade 1 or lower, prior to initiation of crizotinib

### Exclusion Criteria:

Known symptomatic brain metastases
Previous treatment with a PKC inhibitor
Known MSI-H/dMMR tumors who have not previously received immune checkpoint inhibitors
Adverse events from prior anti-cancer therapy that have not resolved
Known acquired immunodeficiency syndrome (AIDS)-related illness, hepatitis B virus, or hepatitis C virus.
Active infection requiring ongoing therapy
Recent surgery or radiotherapy.
Prior gastrectomy or upper bowel removal or any other gastrointestinal disorder or defect
Females who are pregnant or breastfeeding
Impaired cardiac function
Treatment with prohibited medications that cannot be discontinued prior to study entry
For patients receiving IDE 196 powder-in-capsule (PIC) formulation or crizotinib, allergy to mammalian meat products and gelatin.
Prior therapy directly targeting ALK, MET, or ROS1
Spinal cord compression
History of pneumonitis or interstitial lung disease
History of syncope

TABLE 5
Compound 1 + Compound 2 Combination Therapy

| | Compound 1/ | | Selumetinib/ | |
|---|---|---|---|---|
| | Compound 2 | Cabozantinib | Dacarbazine | Tebentafusp |
| Target | PKC/cMET | cMET | MEK/ | HLA-A2-0201 bi- |
| | | | chemotherapy | specific AB |
| Study name | NCT03947385 | Alliance | SUMIT | IMCgp100-102 |
| | | A091201\hat{ } | (NCT01974752) | |
| Population | Any-Line | First-Line | First-Line | Second-Line + |
| | MUM | MUM | MUM | MUM** |
| | (n = 35) | (n = 31) | (n = 97) | (n = 127) |
| Patient | N/A | N/A | N/A | HLA-A2-0201 |
| selection | (100% of | (100% of | (100% of | (~40-50% of |
| | MUM) | MUM) | MUM) | MUM) |
| Drug form | Oral | Oral capsules | Oral capsules | IV infusion |
| | (BID) | (QD) | (BID) and chemo | (weekly) |
| Tolerability | 24% | 51.6% | 63%\hat{ }\hat{ }\hat{ } | 46.5% |
| (Grade ≥3 | | | (all cause) | |
| Drug-related | | | | |
| AE) | | | | |
| % Pts with | 89%* | 23%\hat{ }\hat{ } | 35%\hat{ }\hat{ } | 44%^{#} |
| tumor | | | | |
| shrinkage | | | | |
| Overall | Any-line = 31%/ | 0% | 3%\hat{ }\hat{ }\hat{ } | 4.7%^{#} |
| response | First-line = 50%* | | | |
| rate by | (cPRs only) | | | |
| RECIST 1.1 | | | | |
| Progression | Any-line: ~5 mo./ | 2 mo. | 2.6 mo.\hat{ }\hat{ }\hat{ } | 2.8 mo.^{#} |
| free survival | First-line: >5 mo. | | | |
| (mPFS) | | | | |

^{#}Based on Immunocore reported Second-Line + study data (to reflect comparative patient population) and by independent review.
\hat{ }Randomized Phase II Trial and Tumor Mutational Spectrum Analysis from Cabozantinib versus Chemotherapy in Metastatic Uveal Melanoma (Alliance A091201); Clin Cancer Res 2020; 26: 804-811.
\hat{ }\hat{ }Estimated from waterfall plot.
\hat{ }\hat{ }\hat{ }Journal of Clinical Oncology, Carvajal, et al., 2018; 1232-1239.
*Preliminary analysis of unlocked database as of Jun. 26, 2022 by investigator review.
**Second-line or subsequent-line patients in MUM setting.

Table 5 shows cross-trial comparison with other therapies related to MUM patients. Compound 1 is dosed at 300 mg BID and Compound 2 is dosed at 200 mg BID.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method of treating uveal melanoma or a solid tumor harboring GNAQ or GNA11 mutations in a subject in need thereof, comprising administering to the subject Compound 1: 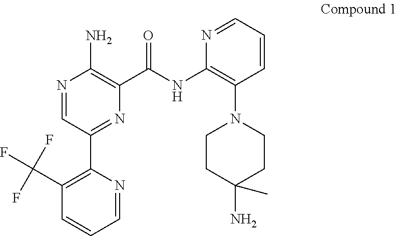 [SMILES: CC1(N)CCN(c2cccnc2NC(=O)c2nc(-c3ncccc3C(F)(F)F)cnc2N)CC1]
or a pharmaceutically acceptable salt thereof,
and Compound 2: 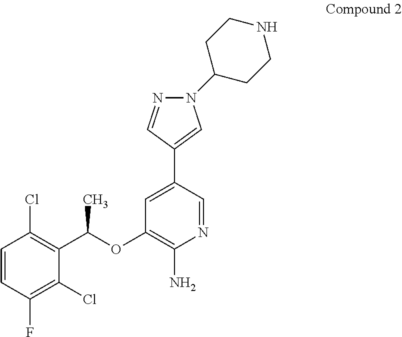 [SMILES: C[C@@H](Oc1cc(-c2cnn(C3CCNCC3)c2)cnc1N)c1c(Cl)ccc(F)c1Cl]
or a pharmaceutically acceptable salt thereof;
wherein
Compound 1, or an equivalent dose of a pharmaceutically acceptable salt thereof, is administered at a dose of about 300 mg two times per day (BID); and
Compound 2, or an equivalent dose of a pharmaceutically acceptable salt thereof, is administered at a dose of about 200 mg BID.

2. A method of treating uveal melanoma or a solid tumor harboring GNAQ or GNA11 mutations in a subject in need thereof, comprising administering to the subject Compound 1: 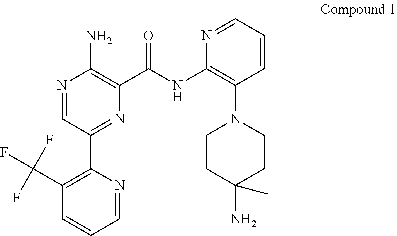 [SMILES: CC1(N)CCN(c2cccnc2NC(=O)c2nc(-c3ncccc3C(F)(F)F)cnc2N)CC1]
or a pharmaceutically acceptable salt thereof,
and Compound 2: 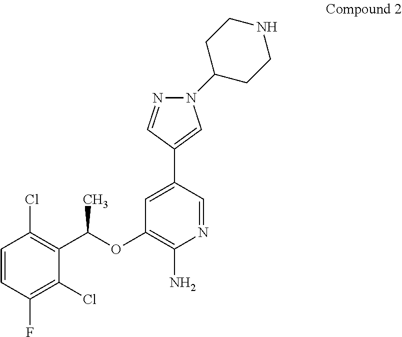 [SMILES: C[C@@H](Oc1cc(-c2cnn(C3CCNCC3)c2)cnc1N)c1c(Cl)ccc(F)c1Cl]
or a pharmaceutically acceptable salt thereof;
wherein
Compound 1, or an equivalent dose of a pharmaceutically acceptable salt thereof, is administered at a dose of about 400 mg to about 600 mg daily; and
Compound 2, or an equivalent dose of a pharmaceutically acceptable salt thereof, is administered at a dose of about 400 mg to about 500 mg daily.

3. A method of treating uveal melanoma or a solid tumor harboring GNAQ or GNA11 mutations in a subject in need thereof, comprising administering to the subject Compound 1: 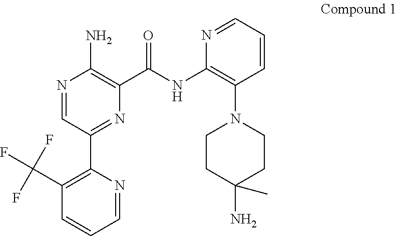 [SMILES: CC1(N)CCN(c2cccnc2NC(=O)c2nc(-c3ncccc3C(F)(F)F)cnc2N)CC1]
or a pharmaceutically acceptable salt thereof,
and Compound 2: 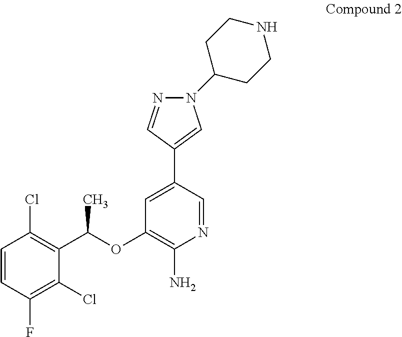 [SMILES: C[C@@H](Oc1cc(-c2cnn(C3CCNCC3)c2)cnc1N)c1c(Cl)ccc(F)c1Cl]
or a pharmaceutically acceptable salt thereof;
(i) according to a dosing schedule comprising a first treatment cycle of at least one 7-day dosing cycle, wherein Compound 1, or an equivalent dose of a pharmaceutically acceptable salt thereof, is administered alone at a dose of about 300 mg BID every day of the first treatment cycle followed by
(ii) a second treatment cycle of at least one 7-day dosing cycle, wherein Compound 1, or an equivalent dose of a pharmaceutically acceptable salt thereof, is administered at a dose of about 300 mg BID, and Compound 2, or an equivalent dose of a pharmaceutically acceptable salt thereof, is administered at a dose of about 200 mg BID every day of the second treatment cycle.

4. A method of treating uveal melanoma or a solid tumor harboring GNAQ or GNA11 mutations in a subject in need thereof, comprising administering to the subject Compound 1: 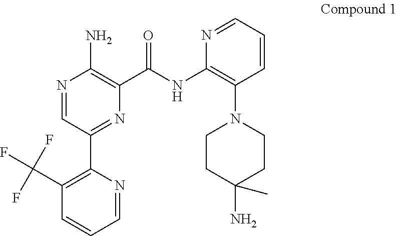 [SMILES: CC1(N)CCN(c2cccnc2NC(=O)c2nc(-c3ncccc3C(F)(F)F)cnc2N)CC1]
or a pharmaceutically acceptable salt thereof,
and Compound 2: 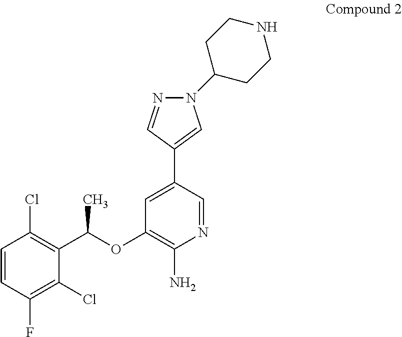 [SMILES: C[C@@H](Oc1cc(-c2cnn(C3CCNCC3)c2)cnc1N)c1c(Cl)ccc(F)c1Cl]
or a pharmaceutically acceptable salt thereof;
according to a dosing schedule comprising at least one 7-day dosing cycle, wherein
Compound 1, or an equivalent dose of a pharmaceutically acceptable salt thereof, is administered at a dose of about 300 mg BID every day of the at least one 7-day dosing cycle; and
Compound 2, or an equivalent dose of a pharmaceutically acceptable salt thereof, is administered at a dose of about 200 mg BID every day of the at least one 7-day dosing cycle.

5. The method of claim 3, wherein the second treatment cycle is two, three, four, five, six, seven, eight, nine, ten, eleven, twelve, thirteen, twenty-four, forty-eight, or ninety-six consecutive 7-day dosing cycles.

6. The method of any one of claims 3 to 5, wherein Compound 1, or a pharmaceutically acceptable salt thereof, and Compound 2, or a pharmaceutically acceptable salt thereof, are co-administered uninterrupted during the at least one 7-day dosing cycle.

7. The method of any one of claims 1 to 6, wherein the uveal melanoma is metastatic uveal melanoma.

8. The method of any one of claims 1 to 7, wherein the uveal melanoma is a solid tumor harboring GNAQ or GNA11 mutations.

9. The method any one of claims 1 to 6, wherein the solid tumor harbors GNAQ or GNA11 mutations.

10. The method of any one of claims 1 to 6 and 9, wherein the solid tumor harboring GNAQ or GNA11 mutations is cutaneous melanoma.

11. The method of any one of claims 1 to 10, wherein Compound 1, or a pharmaceutically acceptable salt thereof, is administered at a dose of 300 mg BID of free base equivalent of Compound 1.

12. The method of any one of claims 1 to 11, wherein Compound 2, or a pharmaceutically acceptable salt thereof, is administered at a dose of 200 mg BID of free base equivalent of Compound 2.

13. The method of any one of claims 1 to 12, wherein Compound 1, or a pharmaceutically acceptable salt thereof, and Compound 2, or a pharmaceutically acceptable salt thereof, are administered in a single formulation.

14. The method of claim 13, wherein Compound 1, or a pharmaceutically acceptable salt thereof, and Compound 2, or a pharmaceutically acceptable salt thereof, are administered in a single formulation further comprising one or more pharmaceutically acceptable carriers.

15. The method of any one of claims 1-12, wherein Compound 1, or a pharmaceutically acceptable salt thereof, and Compound 2, or a pharmaceutically acceptable salt thereof, are administered separately.

16. The method of any one of claims 1-12, and 15, wherein the treatment comprises administering the Compound 1, or a pharmaceutically acceptable salt thereof, and Compound 2, or a pharmaceutically acceptable salt thereof, at substantially the same time.

17. The method of any one of claims 1-12, and 15, wherein the treatment comprises administering Compound 1, or a pharmaceutically acceptable salt thereof, and Compound 2, or a pharmaceutically acceptable salt thereof, at different times.

18. The method of claim 17, wherein Compound 1, or a pharmaceutically acceptable salt thereof, is administered to the subject, followed by administration of Compound 2, or a pharmaceutically acceptable salt thereof.

19. The method of claim 17, wherein Compound 2, or a pharmaceutically acceptable salt thereof, is administered to the subject, followed by administration of Compound 1, or a pharmaceutically acceptable salt thereof.

20. The method of any one of claims 1-19, wherein administering Compound 1, or a pharmaceutically acceptable salt thereof, and Compound 2, or a pharmaceutically acceptable salt thereof, reduces the size of one or more lesions of the uveal melanoma or the solid tumor harboring GNAQ or GNA11 mutations in said subject.

21. The method of any one of claims 1-19, wherein administering Compound 1, or a pharmaceutically acceptable salt thereof, and Compound 2, or a pharmaceutically acceptable salt thereof, decreases the growth rate of one of more lesions of the uveal melanoma or the solid tumor harboring GNAQ or GNA11 mutations in said subject.

22. The method of any one of claims 1-21, comprising administering to the subject in need thereof Compound 1:

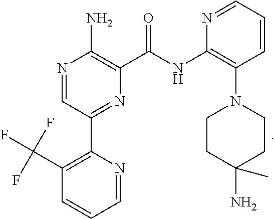 [SMILES: CC1(N)CCN(c2cccnc2NC(=O)c2nc(-c3ncccc3C(F)(F)F)cnc2N)CC1]

23. The method of any one of claims 1-22, comprising administering to the subject in need thereof Compound 2:

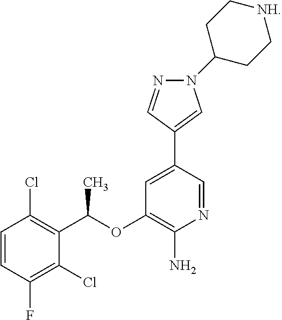 [SMILES: C[C@@H](Oc1cc(-c2cnn(C3CCNCC3)c2)cnc1N)c1c(Cl)ccc(F)c1Cl]

24. The method of any one of claims 1-21, wherein Compound 1, or a pharmaceutically acceptable salt thereof, and Compound 2, or a pharmaceutically acceptable salt thereof, are administered orally.

25. A combination product comprising
(i) about 300 mg Compound 1: 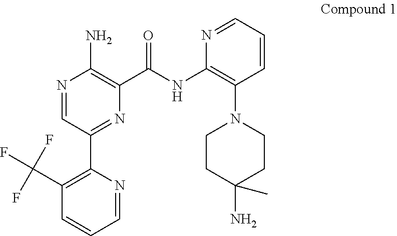 [SMILES: CC1(N)CCN(c2cccnc2NC(=O)c2nc(-c3ncccc3C(F)(F)F)cnc2N)CC1]
or an equivalent dose of a pharmaceutically acceptable salt thereof, and
(ii) about 200 mg of Compound 2: 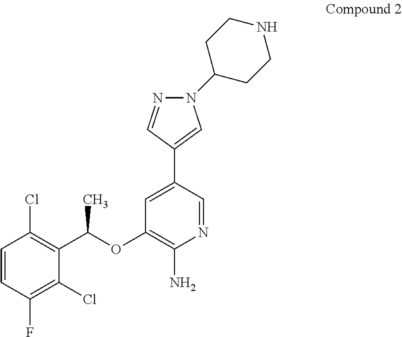 [SMILES: C[C@@H](Oc1cc(-c2cnn(C3CCNCC3)c2)cnc1N)c1c(Cl)ccc(F)c1Cl]
or an equivalent dose of a pharmaceutically acceptable salt thereof.

26. A combination product comprising
(i) about 200 mg to about 300 mg Compound 1: 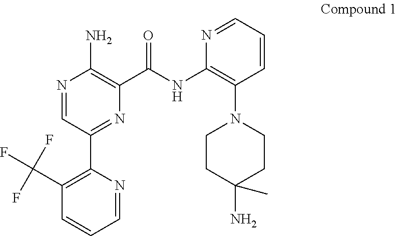 [SMILES: CC1(N)CCN(c2cccnc2NC(=O)c2nc(-c3ncccc3C(F)(F)F)cnc2N)CC1]
or an equivalent dose of a pharmaceutically acceptable salt thereof, and
(ii) about 200 mg to about 250 mg of Compound 2: 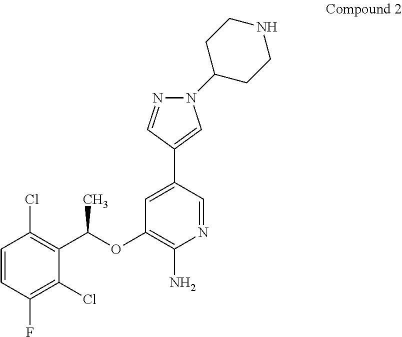 [SMILES: C[C@@H](Oc1cc(-c2cnn(C3CCNCC3)c2)cnc1N)c1c(Cl)ccc(F)c1Cl]
or an equivalent dose of a pharmaceutically acceptable salt thereof.

27. The combination product of claim 26, wherein the combination product comprises 300 mg of Compound 1, or an equivalent dose of a pharmaceutically acceptable salt thereof.

28. The combination product of claim 26 or 27, wherein the combination product comprises 200 mg of Compound 2, or an equivalent dose of a pharmaceutically acceptable salt thereof.

29. A method of treating uveal melanoma or a solid tumor harboring GNAQ or GNA11 mutations in a subject in need thereof, comprising administering to the subject Compound 1 or a pharmaceutically acceptable salt thereof, and Compound 2, or a pharmaceutically acceptable salt thereof;
wherein
Compound 1, or an equivalent dose of a pharmaceutically acceptable salt thereof, is administered at a dose of about 200 mg two times per day (BID); and
Compound 2, or an equivalent dose of a pharmaceutically acceptable salt thereof, is administered at a dose of about 200 mg BID.

30. The method of claim 29, wherein Compound 1, or a pharmaceutically acceptable salt thereof, is administered at a dose of 200 mg BID of free base equivalent of Compound 1.

31. The method of claim 29 or 30, wherein Compound 2, or a pharmaceutically acceptable salt thereof, is administered at a dose of 200 mg BID of free base equivalent of Compound 2.

32. A method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;
wherein
Compound 1, or an equivalent dose of a pharmaceutically acceptable salt thereof, is administered at a dose of about 300 mg two times per day (BID); and
Compound 2, or an equivalent dose of a pharmaceutically acceptable salt thereof, is administered at a dose of about 250 mg BID.

33. The method of claim 32, wherein Compound 1, or a pharmaceutically acceptable salt thereof, is administered at a dose of 300 mg BID of free base equivalent of Compound 1.

34. The method of claim 32 or 33, wherein Compound 2, or a pharmaceutically acceptable salt thereof, is administered at a dose of 250 mg BID of free base equivalent of Compound 2.

35. The method of any one of claims 29 to 34, wherein the uveal melanoma is metastatic uveal melanoma.

36. The method of any one of claims 29 to 35, wherein the uveal melanoma is a solid tumor harboring GNAQ or GNA11 mutations.

37. The method of any one of claims 29 to 34, wherein the solid tumor harbors GNAQ or GNA11 mutations.

38. The method of any one of claims 29 to 34 and 37, wherein the solid tumor harboring GNAQ or GNA11 mutations is cutaneous melanoma.

39. The method of any one of claims 29 to 38, wherein Compound 1, or a pharmaceutically acceptable salt thereof, and Compound 2, or a pharmaceutically acceptable salt thereof, are administered in a single formulation.

40. The method of claim 39, wherein Compound 1, or a pharmaceutically acceptable salt thereof, and Compound 2, or a pharmaceutically acceptable salt thereof, are administered in a single formulation further comprising one or more pharmaceutically acceptable carriers.
